(19) United States
(12) Patent Application Publication
(10) Pub. No.: US 2026/0232879 A1
(43) Pub. Date: Aug. 13, 2026

(54) RETAINING DEVICE FOR MOUNTING A SENSOR ELEMENT TO A DIALYSIS MACHINE

(71) Applicant: B. Braun Avitum AG, Melsungen (DE)

(72) Inventor: Sebastian Horschke, Hessisch Lichtenau (DE)

(21) Appl. No.: 19/530,727

(22) Filed: Feb. 5, 2026

(30) Foreign Application Priority Data
Feb. 11, 2025 (DE) 10 2025 104 880.0

(51) Int. Cl. A61M 1/14

(52) U.S. Cl. CPC A61M 1/14; A61M 2205/3317; A61M 2209/082

(57) ABSTRACT

A retaining device is configured to mount a sensor element to a dialysis machine. The retaining device includes a fastening portion for fastening the retaining device to the dialysis machine, and a hollow cylindrical portion extending between a first hollow cylinder end and a second hollow cylinder end. The hollow cylindrical portion has an axial retaining bore and an insertion slit. The first hollow cylinder end is connected to the fastening portion. The retaining bore is adapted for receiving the sensor element. The insertion slit extends between the first hollow cylinder end and the second hollow cylinder end and forms a C-shaped cross section. The hollow cylindrical portion is open in the circumferential direction, and the insertion slit leads radially into the retaining bore. The hollow cylindrical portion is resiliently flexible so that the sensor element can be introduced radially into the retaining bore through the insertion slit.

### CROSS-REFERENCE TO RELATED APPLICATIONS

This application claims priority under 35 U.S.C. § 119 to German Application No. 10 2025 104 880.0, filed on Feb. 11, 2025, the content of which is incorporated by reference herein in its entirety.

### FIELD

The present disclosure relates to a retaining device for mounting a sensor element to a dialysis machine.

### BACKGROUND

Dialysis machines are conventionally equipped with sensor elements, in particular with reed sensors which are also referred to as reed switches.

It is an object of the present disclosure to enable simplified fitting and removal of a sensor element on a dialysis machine.

The retaining device according to the present disclosure is intended for mounting a sensor element, preferably a reed sensor, to a dialysis machine, and comprises a fastening portion and a hollow cylindrical portion. The fastening portion is adapted for fastening the retaining device to the dialysis machine. The hollow cylindrical portion extends along a longitudinal axis between a first hollow cylinder end and a second hollow cylinder end. The hollow cylindrical portion comprises an axial retaining bore and an insertion slit. The first hollow cylinder end is connected to the fastening portion. The retaining bore is adapted for receiving the sensor element. The insertion slit extends continuously between the first hollow cylinder end and the second hollow cylinder end so as to form a C-shaped cross section of the hollow cylindrical portion, which is open in the circumferential direction. The insertion slit leads radially into the retaining bore. The hollow cylindrical portion is resiliently flexible at least in portions. The sensor element can therefore be introduced radially into the retaining bore through the insertion slit. The retaining device according to the present disclosure enables simplified fitting and removal of the sensor element. For the purpose of fitting, the retaining device is initially fastened to the dialysis machine by means of the fastening portion. The sensor element is inserted radially into the retaining bore through the insertion slit. Since the hollow cylindrical portion is resiliently flexible at least in portions, the insertion slit yields resiliently during the insertion of the sensor element under the effect of the latter, expands in the circumferential direction and springs back resiliently after the insertion of the sensor element. In the inserted state, the sensor element is retained in the retaining bore by a force fit and/or form fit. In the inserted state, the hollow cylindrical portion forms a latching, clamping and/or snap-fit connection to the sensor element. The removal may take place in the kinematically opposite way.

In one embodiment of the present disclosure, the retaining bore is open axially on both sides and, in the region of the second hollow cylinder end, comprises at least one projection which protrudes radially inwards. The at least one projection protruding radially inwards prevents, or at least hinders, axial extraction of the sensor element from the retaining bore. By configuring the retaining bore to be open on both sides, any signal line of the sensor element can be fed out at one end from the retaining bore in a particularly straightforward way. Configuring the retaining bore to be open at the other end as well can enable an improved function of the sensor element.

In a further embodiment of the present disclosure, the hollow cylindrical portion comprises resiliently flexible clamping portions in the region of the second hollow cylinder end, which protrude laterally into the insertion slit and are adapted for resilient clamping of the sensor element. The clamping portions so to speak form end portions of the C-shaped cross section and lie opposite one another in the circumferential direction. The insertion slit is narrower in the region of the clamping portions than away from the clamping portions. The clamping portions form a partial boundary of the insertion slit. The clamping portions may also be referred to as clamping edges of the insertion slit. In one embodiment, each of the clamping portions extends over 20% to 80%, preferably over 30% to 60%, more preferably over 40 to 50%, of a total length of the hollow cylindrical portion.

In a further embodiment of the present disclosure, the insertion slit comprises a widening in the region of the first hollow cylinder end and therefore in the region of the fastening portion. The widening, which may also be referred to as a clearance, avoids contact between the sensor element and the insertion slit (or more precisely edges of the hollow cylindrical portion which border the insertion slit) in the region of the first hollow cylinder end, and therefore also in the region of the fastening portion, when the sensor element is being introduced into the retaining bore. Mechanical stress peaks and therefore mechanical overloading of the hollow cylindrical portion are therefore avoided in the region of the widening.

In a further embodiment of the present disclosure, the hollow cylindrical portion and the fastening portion are connected integrally to one another. The integral connection obviates the need for a separate joining connection and corresponding joining means for connecting the hollow cylindrical portion to the fastening portion. A particularly simple structure of the retaining device is thereby achieved. Preferably, the retaining device is formed in one piece.

In a further embodiment of the present disclosure, the retaining device is made in one piece from plastic. In one embodiment, the retaining device is an injection-moulded part made from plastic. In a further embodiment, it is made by additive manufacturing, for example by means of 3D printing. In one embodiment, the plastic used is polyoxymethylene (POM). In a further embodiment, the plastic used is polyamide (PA).

In a further embodiment of the present disclosure, the hollow cylindrical portion is attached to the fastening portion by means of ribbing. The ribbing provides mechanical stiffening and counteracts unintended resilient deformations. The ribbing may in principle be configured in any way which is suitable for the present purpose.

In a further embodiment of the present disclosure, the ribbing comprises a plurality of ribs, each of which extends axially starting from the fastening portion as far as an outer surface of the hollow cylindrical portion. In other words: each of the ribs is connected at one end to the fastening portion and at the other end to the hollow cylindrical portion. Preferably, each of the ribs extends along the longitudinal axis of the hollow cylindrical portion, in particular parallel to the longitudinal axis.

In a further embodiment of the present disclosure, the ribbing comprises a plurality of ribs which are arranged mutually offset in the circumferential direction of the hollow cylindrical portion. Preferably, each of the plurality of ribs — in accordance with the preceding embodiment — extends axially starting from the fastening portion as far as an outer surface of the hollow cylindrical portion. Arranging the plurality of ribs offset in the circumferential direction offers advantageous mechanical properties.

In a further embodiment of the present disclosure, the hollow cylindrical portion is attached to the fastening portion by means of a rounding. The rounding is arranged in the region of the first hollow cylinder end. The rounding counteracts a notch effect and therefore mechanical stress peaks. Because of the rounding, the hollow cylindrical portion has a larger outer diameter in the region of the first hollow cylinder end than in the region of the second hollow cylinder end.

In a further embodiment of the present disclosure, the ribbing is arranged on the rounding. Arranging the ribbing on the rounding achieves a configuration of the retaining device which is particularly advantageous in mechanical terms.

In a further embodiment of the present disclosure, the fastening portion comprises at least one flange portion, which protrudes radially from the second hollow cylinder end and which has at least one fastening bore for receiving a fastening means. Preferably, the at least one flange portion is arranged orthogonally with respect to the longitudinal axis of the hollow cylindrical portion. Preferably, the fastening bore is adapted for receiving a fastening screw. In one preferred embodiment, the fastening portion comprises two flange portions which are arranged on radially opposite sides. In other words: the two flange portions are arranged mutually offset by 180° about the longitudinal axis of the hollow cylindrical portion.

The present disclosure also relates to an arrangement having a retaining device according to preceding description and having a sensor element, which is retained in the retaining bore of the retaining device.

The present disclosure also relates to a dialysis machine having a retaining device according to the preceding description. Alternatively or in addition, the dialysis machine according to the present disclosure comprises an arrangement according to the preceding embodiment.

### BRIEF DESCRIPTION OF THE DRAWINGS

Further advantages and features of the present disclosure may be found in the following description of a preferred exemplary embodiment of the present disclosure, which is represented with the aid of the drawings, in which:

FIG. 1 shows a schematic perspective view of an embodiment of a dialysis machine according to the present disclosure comprising an embodiment of a retaining device according to the present disclosure;

FIG. 2 shows a schematic perspective view of the retaining device of the dialysis machine according to FIG. 1; and

FIG. 3 shows a schematic longitudinal section of the retaining device according to FIG. 2 together with a sensor element in the form of a reed sensor, which is retained by means of the retaining device.

### DETAILED DESCRIPTION

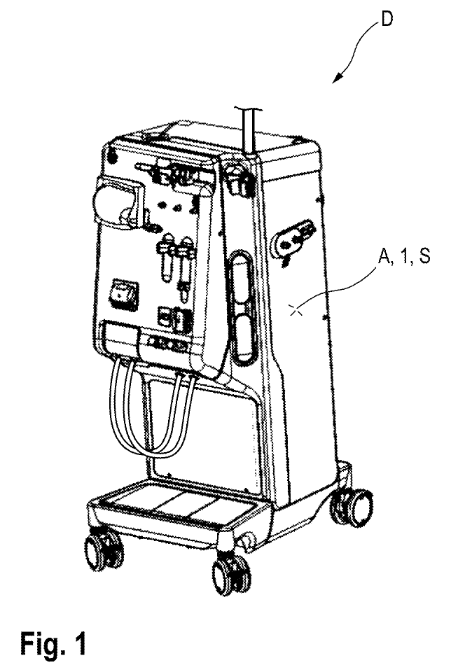

According to FIG. 1, a dialysis machine D for extracorporeal blood treatment is provided. The basic structure and the function of the dialysis machine D are known to a person skilled in the art. Further details in this regard need not therefore be discussed at length.

The dialysis machine D is equipped with a sensor element S, which is retained and fastened on the machine side by means of a retaining device 1.

The retaining device 1 and the sensor element S together form an arrangement A.

The arrangement A, which is not shown in detail in FIG. 1, is arranged at a place intended for it inside the dialysis machine D. The actual positioning of the arrangement A in relation to the dialysis machine D is not of great importance for the present disclosure. To this extent, the exemplary positioning of the arrangement A behind a side wall of a housing of the dialysis machine D as shown in FIG. 1 is to be understood as merely exemplary.

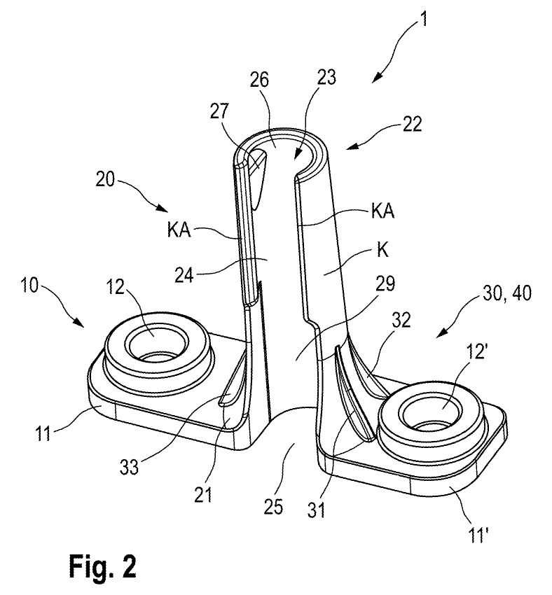

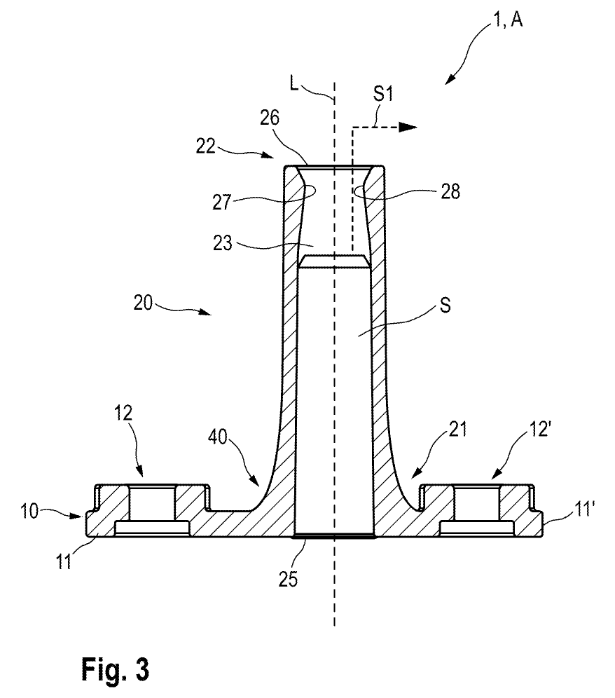

The retaining device 1 is shown in detail in FIG. 2 and 3; it comprises a fastening portion 10 and a hollow cylindrical portion 20.

The fastening portion 10 is adapted for fastening the retaining device 1 to the dialysis machine D. The fastening portion 10 has a specific structure, which is described in more detail below.

The hollow cylindrical portion 20 extends along a longitudinal axis L (see FIG. 3) between a first hollow cylinder end 21 and a second hollow cylinder end 22. The hollow cylindrical portion 20 comprises a retaining bore 23 and an insertion slit 24.

In the embodiment shown, the first hollow cylinder end 21 is connected to the fastening portion 10. This connection is formed in a manner which is explained in more detail below.

The retaining bore 23 is configured for receiving the said sensor element S. The received state is shown schematically in FIG. 3.

In the embodiment shown, the sensor element S is a reed sensor. In the embodiment shown, the sensor element S has a cylindrical design. An outer contour of the sensor element S is configured to match an inner contour of the hollow cylindrical portion 20 and/or of the retaining bore 23. In the received state, the sensor element S is received coaxially with respect to the longitudinal axis L in the retaining bore 23. An outer diameter of the sensor element S corresponds approximately to an inner diameter of the retaining bore 23. In particular, the outer diameter of the sensor element S is slightly greater than the inner diameter of the retaining bore 23.

The insertion slit 24 extends continuously between the first hollow cylinder end 21 and the second hollow cylinder end 22 so as to form a C-shaped cross section of the hollow cylindrical portion 20, which is open in the circumferential direction. The insertion slit 24 leads radially, and therefore orthogonally with respect to the longitudinal axis L, into the retaining bore 23.

The hollow cylindrical portion 20 is resiliently flexible at least in portions. The sensor element S can therefore be inserted radially into the retaining bore 23 through the insertion slit 24. In other words: the sensor element S can be latched or snap-fastened into the retaining bore 23 and therefore into the hollow cylindrical portion 20. This greatly simplifies the fitting (and removal) of the sensor element S on the dialysis machine D.

In the embodiment shown, the hollow cylindrical portion 20 is open axially on both sides. Accordingly, the retaining bore 23 has a first end opening 25 in the region of the first hollow cylinder end 21. In the region of the second hollow cylinder end 22, the retaining bore 23 has a second end opening 26. The two end openings 25, 26 lie opposite one another along the longitudinal axis L.

In the exemplary configuration shown in FIG. 3, a lower end face of the sensor element S is arranged approximately flush with the first end opening 25. An opposite end face of the sensor element S is set back axially from the second end opening 26.

In order to prevent unintended axial extraction of the sensor element S through the second end opening 26, the retaining bore 23 comprises at least one projection 27, 28 in the region of the second hollow cylinder end 22. The at least one projection 27, 28 protrudes radially inwards. An inner diameter of the retaining bore 23 is therefore narrowed in the region of the second hollow cylinder end 22.

In the present case, an inner diameter of the retaining bore 23 in the region of the at least one projection 27, 28 is less than the outer diameter of the sensor element, in such a way that even resilient deformation of the hollow cylindrical portion 20 does not allow axial extraction of the sensor element S from the second end opening 26.

There are two projections in the embodiment shown, which may also be referred to as the first projection 27 and the second projection 28. The two projections 27, 28 are arranged mutually offset about the longitudinal axis L and therefore in the circumferential direction. The offset in the present case is 180°.

In the embodiment shown, the sensor element S comprises a signal line S1. The signal line S1 is connected, preferably non-releasably, at one end to the sensor element S. At the other end, the signal line S1 can be attached to a fixture of the dialysis machine D, which is intended for it. In the embodiment shown, the signal line S1 is fed out from the hollow cylindrical portion 20 through the second end opening 26.

In the embodiment shown, the hollow cylindrical portion 20 comprises resiliently flexible clamping portions KA in the region of the second hollow cylinder end 22, which protrude laterally into the insertion slit 24 and are adapted for resilient clamping of the sensor element S. The insertion slit 24 is narrower in the region of the clamping portions KA than away from the clamping portions KA. The clamping portions KA form a partial boundary of the insertion slit 24. The clamping portions KA may also be referred to as clamping edges. In the embodiment shown, each of the clamping portions KA extends over 50% of a total length of the hollow cylindrical portion 20.

In the embodiment shown, the insertion slit 24 comprises a widening 29 in the region of the first hollow cylinder end 21 and therefore in the region of the fastening portion 10. The widening 29 may also be referred to as a clearance. In the embodiment shown, the widening 29 extends approximately over one half of a total length of the hollow cylindrical portion 20. The widening 29 avoids stress peaks during the radial latching of the sensor element S into the retaining bore 23.

In the embodiment shown, the hollow cylindrical portion 20 and the fastening portion 10 are connected integrally to one another. In particular, the entire retaining device 1 is formed in one piece.

In the present case, the retaining device 1 is made in one piece from plastic K. Polyoxymethylene (POM) and/or polyamide (PA) may particularly be envisaged as the plastic K. In the embodiment shown, the retaining device 1 is made by injection moulding. Alternatively, in an embodiment which is not shown in the figures, it is made by additive manufacturing, for example by means of 3D printing.

In the embodiment shown, the hollow cylindrical portion 20 is attached to the fastening portion 10 by means of ribbing 30. The ribbing 30 provides mechanical stiffening. The ribbing 30 is formed in the region of the first hollow cylinder end 21.

In the embodiment shown, the ribbing 30 comprises a plurality of ribs 31, 32, 33, only three ribs of the plurality of ribs being visible in FIG. 2 because of the perspective shown there. Each of the plurality of ribs 31, 32, 33 extends axially starting from the fastening portion 10 as far as an outer surface of the hollow cylindrical portion 20. In the embodiment shown, the longitudinal extent of the ribs 31, 32, 33 is parallel to the longitudinal axis L of the hollow cylindrical portion 20.

Each of the plurality of ribs 31, 32, 33 is connected at one end to the fastening portion 10 and at the other end to the hollow cylindrical portion 20.

In the embodiment shown, the plurality of ribs 31, 32, 33 are arranged mutually offset in the circumferential direction of the hollow cylindrical portion 20 and therefore about the longitudinal axis L. The offset in the present case is uniform.

In the present case, the hollow cylindrical portion 20 is attached to the fastening portion 10 by means of a rounding 40. The rounding 40 extends circumferentially in the region of the first hollow cylinder end 21 and reduces a notch effect between the hollow cylindrical portion 20 and the fastening portion 10. Because of the rounding 40, the hollow cylindrical portion 20 has a design at one end which widens in a similar way to a tree trunk in the region of the roots.

In the embodiment shown, the ribbing 30 is arranged on the rounding 40. The resulting configuration is particularly advantageous, and particularly capable of bearing a load.

In the embodiment shown, the fastening portion 10 comprises at least one flange portion 11. In particular, in the present case there are two flange portions 11, 11’, which may also be referred to as the first flange portion 11 and the second flange portion 11’. Each of the two flange portions 11, 11’ protrudes radially from the first hollow cylinder end 21, in particular orthogonally with respect to the longitudinal axis L.

The two flange portions 11, 11’ in the present case are arranged mutually offset about the longitudinal axis L. The offset in the present case is 180°.

Each of the two flange portions 11, 11’ comprises a fastening bore 12, 12’, the latter being adapted for receiving a fastening means. In the present case, fastening screws are provided as the fastening means.

In the fastened state of the retaining device 1, the two flange portions 11, 11’ lie flat on a portion of the dialysis machine D, which is intended for them, and are screwed to the said portion by means of the said fastening screws.

1. A retaining device for mounting a sensor element to a dialysis machine, the retaining device comprising:
a fastening portion adapted for fastening the retaining device to the dialysis machine; and
a hollow cylindrical portion extending along a longitudinal axis between a first hollow cylinder end and a second hollow cylinder end,
the hollow cylindrical portion having an axial retaining bore and an insertion slit,
the first hollow cylinder end being connected to the fastening portion,
the axial retaining bore being adapted for receiving the sensor element,
the insertion slit extending continuously between the first hollow cylinder end and the second hollow cylinder end so as to form a C-shaped cross section of the hollow cylindrical portion, the hollow cylindrical portion being open in a circumferential direction of the hollow cylindrical portion, and the insertion slit leading radially into the axial retaining bore, and
the hollow cylindrical portion being resiliently flexible, at least in portions, to permit the sensor element to be introduced radially into the axial retaining bore through the insertion slit.

2. The retaining device according to claim 1, wherein:
the axial retaining bore is open axially at the first hollow cylinder end and at the second hollow cylinder end,
the axial retaining bore comprises at least one projection in an area of the second hollow cylinder end, and
the at least one projection protrudes radially inwardly to counteract axial extraction of the sensor element from the axial retaining bore.

3. The retaining device according to claim 1, wherein:
the hollow cylindrical portion comprises resiliently flexible clamping portions in an area of the second hollow cylinder end,
the resiliently flexible clamping portions protrude laterally into the insertion slit, and
the resiliently flexible clamping portions are adapted for resilient clamping of the sensor element.

4. The retaining device according to claim 1, wherein the insertion slit comprises a widening in an area of the first hollow cylinder end and in an area of the fastening portion.

5. The retaining device according to claim 1, wherein the hollow cylindrical portion and the fastening portion are connected integrally to one another.

6. The retaining device according to claim 5, wherein the retaining device is a one-piece homogeneous body of unitary construction formed of plastic.

7. The retaining device according to claim 5, wherein the hollow cylindrical portion is attached to the fastening portion by ribbing.

8. The retaining device according to claim 7, wherein:
the ribbing comprises a plurality of ribs,
each of the plurality of ribs extends axially from the fastening portion to an outer surface of the hollow cylindrical portion.

9. The retaining device according to claim 7, wherein:
the ribbing comprises a plurality of ribs,
the plurality of ribs are arranged mutually offset in the circumferential direction of the hollow cylindrical portion.

10. The retaining device according to claim 9, wherein:
the hollow cylindrical portion is attached to the fastening portion by rounding, and
the ribbing is arranged on the rounding.

11. The retaining device according to claim 1, wherein the hollow cylindrical portion is attached to the fastening portion by rounding.

12. The retaining device according claim 1, wherein:
the fastening portion comprises at least one flange portion,
the at least one flange portion protrudes radially from the first hollow cylinder end, and
the at least one flange portion has at least one fastening bore for receiving a fastener.

13. A dialysis machine comprising the retaining device according to claim 1.

14. An arrangement comprising:
the retaining device according to claim 1; and
a sensor element retained in the axial retaining bore of the retaining device.

15. A dialysis machine comprising the arrangement according to claim 14.
